(19) United States
(12) Patent Application Publication
(10) Pub. No.: US 2005/0283092 A1
(43) Pub. Date: Dec. 22, 2005

(54) Continuous compartment pressure monitoring device

(75) Inventor: Tewodros Gedebou, Los Angeles, CA (US)

Correspondence Address: PILLSBURY WINTHROP SHAW PITTMAN LLP, 725 S. FIGUEROA STREET, SUITE 2800, LOS ANGELES, CA, 90017, US

(73) Assignee: Cedars-Sinai Medical Center

(21) Appl. No.: 10/873,851

(22) Filed: Jun. 22, 2004

(51) Int. Cl. A61B 5/00

(52) U.S. Cl. 600/561

(57) ABSTRACT

Described herein is a compartment pressure monitoring device that enables the continuous monitoring of pressure in a physiological compartment, such as those found in the hand, forearm, upper arm, abdomen, pericardium, thigh and leg. In various embodiments, the device includes a tube connected at a first end to a balloon, and at a second end to a pressure gauge. A trocar sleeve may also be connected to the tube at or near its first end, such that a compatible trocar may be used to facilitate the insertion and positioning of the device within the compartment. The pressure gauge includes an alarm device configured to sound or display an alarm when a predetermined pressure threshold on the balloon has been met or exceeded. Also described is a method of continuously monitoring compartment pressure, using the compartment pressure monitoring device of the invention.

### FIELD OF INVENTION

The invention relates to a physiological compartment pressure-monitoring device. In particular embodiments, the invention relates to a continuous compartment pressure-monitoring device that may be inserted directly into the compartment of a patient, and may also include a pressure gauge with an alarm to indicate if a predetermined pressure threshold has been reached.

### BACKGROUND OF THE INVENTION

Compartment syndrome is a painful condition that results when pressure within muscle tissue reaches unsafe levels; preventing nutrients, such as glucose and oxygen, from reaching nerve and muscle cells. Compartments are located in anatomical locations throughout the body of a mammal. For example, compartments may be found in the hand, forearm, upper arm, abdomen, buttock and leg. Compartment syndrome can be acute or chronic. Acute compartment syndrome can have severe consequences, such as paralysis, loss of limb or death. It can affect, for example, the cardiovascular, renal or pulmonary health of a patient. While generally not a medical emergency, chronic compartment syndrome poses significant problems as well, and frequently presents in athletes.

The type of swelling that leads to compartment syndrome is often associated with high-energy trauma, such as from a car accident or crush injury, or from burns, electric injury, cold injury, etc. Surgical causes are also common (e.g., from post operative hemorrhaging, orthopedics, vascular surgery or implementation of laparoscopic techniques). Compartment syndrome can also result from the application of tight bandages and casts, lying on limb, excessive traction on fractured limbs, closure of fascial defects, increased capillary filtration, leaking dialysis catheter, high pressure injections, acute rhabdomyolysis or nephritic syndrome. Other causes of compartment syndrome are intense exercise, seizures, tetany, the repeated use of a specific muscle group, snakebites, anabolic steroid use and swelling of the muscle itself. Pressure of abdominal packs and edema of the bowel or retroperitoneum may contribute to increased intra-abdominal pressure (“LAP”). Bleeding into a cavity like the pericardial pouch following surgery or trauma may also result in pericardial tamponade, squeezing the heart.

Prompt diagnosis of compartment syndrome may help ensure a successful outcome for a patient. Often, immediate surgical intervention is required to avoid organ failure, limb loss and, possibly, death. Current diagnostic methods for compartment syndrome include simple; periodic examination by a physician (i.e., by touch and an assessment of patient response to pain) and/or static measurement of compartment pressure, which requires repeated examinations and/or punctures. These diagnostic methods may incorporate the use of an ultrasound probe, tonometer, slit catheter or wick to measure compartment pressure.

### SUMMARY OF THE INVENTION

Embodiments of the present invention are directed to a compartment pressure monitoring device and methods of using the same. Further embodiments of the present invention are directed to a compartment pressure-monitoring device that includes a tube, connected at a first end to a balloon, and at a second end to a pressure gauge. A trocar sleeve may be further connected to the tube at or near its first end. The pressure gauge may include an alarm device configured to sound or display an alarm when a predetermined pressure threshold (measured on the balloon) has been met or exceeded. By way of example, for the abdominal compartment, the predetermined pressure threshold may be about 30 to 40 mmHg.

Still further embodiments of the present invention are directed to a compartment pressure-monitoring device used in combination with a trocar. The trocar may be used to facilitate insertion and/or placement of the device within a compartment, and may be removed once the device is satisfactorily inserted and/or placed.

Yet further embodiments of the present invention are directed to a compartment pressure-monitoring device including a rigid support member attached to the tube. A syringe may additionally be attached to the tube by way of a secondary access tube with a standard fitting, such as a Luer lock type fitting; thereby providing fluid access to the tube. Various fluids may thus be introduced or removed from the inner portion of the tube.

### BRIEF DESCRIPTION OF THE FIGURES

FIG. 1 illustrates a compartment pressure monitoring device, in accordance with an embodiment of the present invention.

FIG. 2 illustrates a compartment pressure monitoring device implanted in a human leg compartment, in accordance with an embodiment of the present invention.

### DETAILED DESCRIPTION OF THE INVENTION

The invention relates to a medical device that may be used to treat and/or prevent compartment syndrome. Treatment and/or prevention, as used herein, may include, but are in no way limited to, lessening the severity of the disease condition, preventing the disease condition from occurring, preventing the disease condition from worsening, curing the disease condition, prolonging a patient's life or life expectancy or any other therapeutic attempt to address the disease condition, even if such attempt is ultimately unsuccessful.

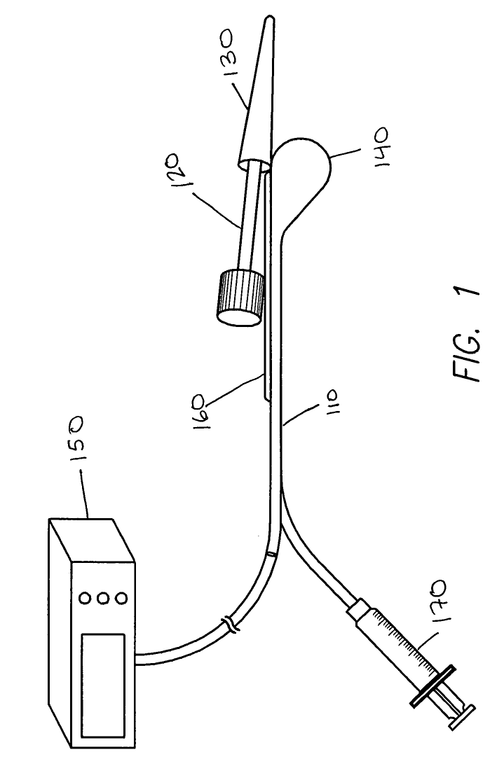

As depicted in FIG. 1, the device 100 may include a tube 110; a portion of which may be configured to be inserted into a region of a compartment within a patient in which compartment syndrome is prone to develop (i.e., the “first end” of the tube), and another portion of which may be configured to remain exterior to the body when the device is in use (i.e., the “second end” of the tube). Examples of such compartments include compartments in the human hand, forearm, upper arm, abdomen, pericardium, thigh and leg, although the device 100 may be suitable for use in connection with other compartments, as well. The tube 110 may be constructed of any conventional medical grade material, as will be readily appreciated by those of skill in the art; for example, various plastics may be used to construct the tube 110 of the invention, as well as alloys, silicone and the like. The device 100 may further include a trocar 120 and trocar sleeve 130 to guide the tube 110 into a patient's compartment. Once the tube 110 is inserted into the patient's compartment, the trocar 120 may be removed, leaving the trocar sleeve 130 inside the compartment, attached to the tube 110. The device 100 may alternately or additionally include any other type of guide to insert and/or position the tube 110 within the patient's compartment.

At one end of the tube 110 (the end that is adapted to be located within a patient's compartment) resides a balloon 140. As used herein, the term “balloon” is intended to encompass any similar apparatus that may mechanically deform in response to local pressure exerted upon its exterior. The other end of the tube 110 is connected to a pressure gauge or transducer 150. If local pressure in a patient's compartment rises above a predetermined pressure threshold, as determined by the balloon/pressure gauge assembly, an alarm is activated to alert of the pressure rise. More particularly, when the pressure in the compartment rises, it will increase the atmospheric forces on the exterior surface of the balloon 140. This, in turn, pushes fluid 190 contained within the balloon 140 through the tube 110, affecting the measurement of the pressure gauge 150. The alarm may be visual, auditory, tactile or any other type of alarm. The alarm may also use more than one type of signal, for example auditory and visual, in combination or alternatively.

The pressure threshold may be set to any appropriate level, depending upon the physiological characteristics of the patient, the compartment being monitored, and any number of other factors that will readily be appreciated by those of skill in the art. By way of example, in monitoring IAP, an appropriate pressure threshold may be about 30 mmHg, and, in some cases, 40 mmHg may be sufficient for an extremity to survive. In patients with low blood pressure, the parameter of 20 mmHg below the diastolic pressure may be used. Additionally, depending on the patient's clinical conditions, the threshold may be altered appropriately (for example, alarming if a mean pressure of greater than 30 mmHg is noted over a given period, or if the mean pressure is rising at a given rate).

A rigid support member 160 may be included along the tube 110 to facilitate insertion into the compartment by maintaining the rigidity of the tube 110 during insertion and positioning. Similar to the tube 110, the rigid support member 160 may be constructed of any suitable medical grade material, such as various types of plastic, alloys, silicone and the like. Any conventional means may be employed to affix the rigid support member 160 to the tube 110. Moreover, although rigid in nature, the support member 160 may possess some degree of pliability, to avoid its undesirable breakage during insertion/positioning or thereafter, once the device 100 of the invention has been implanted within a patient and compartment pressure is being monitored.

The tube 110 may also include a fitting 180 (e.g., a Luer lock fitting) that enables fluid access by a syringe 170 or similar apparatus to the interior of the tube 110. The fitting may be configured upon an access tube 185 that connects with the tube 110. As such, the syringe 170 may be configured as a Luer lock syringe, or as any other suitable type of syringe or similar apparatus, depending upon the configuration of the fitting 180. The syringe 170 may thus be detachable from the tube 110. The syringe 170 provides for the introduction and removal of fluid 190 from the interior of the tube 110. The fluid 190 may be, for example, water, saline or any other conventional fluid appropriate for use in connection with the device 100 of the present invention to measure pressure. The fluid 190 enables the assessment of internal physiological pressure when the tube 110 is in place in a cavity.

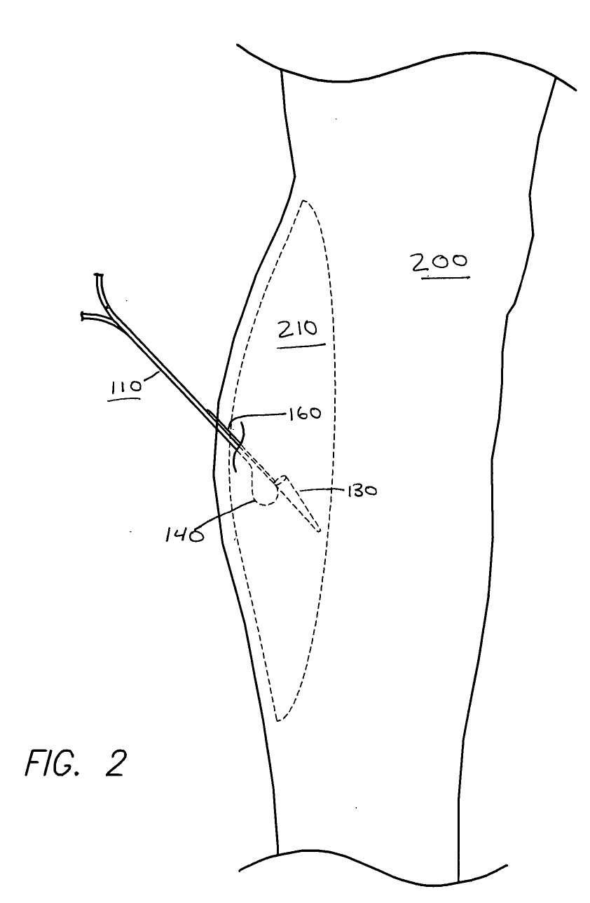

FIG. 2 illustrates an embodiment of the invention in which the device has been inserted into a human leg compartment 200. This embodiment illustrates but one example of a compartment wherein the device 100 of the present invention may be used to monitor internal physiological pressure. A tiny surgical incision or puncture is made to enable surgical access to the interior of the compartment sought to be monitored with the device 100; the trocar 120 may thereafter be used to insert and position the first end of the device 100 within the compartment. As depicted in FIG. 2, the trocar 120 has been removed, leaving the trocar sleeve 130, rigid support member 160 and a portion of the tube 110 (with the balloon 140) within the compartment 210. When pressure increases inside the compartment 210, the balloon 140 is compressed from its exterior by the local atmospheric pressure, thereby forcing fluid 190 through the tube 110 toward the pressure gauge 150, causing the alarm therein to activate.

The device 100 of the present invention allows a physician, a health care worker or any other person to continuously monitor compartment pressure in a mammal. It may be used before, during or after a surgical operation or other form of medical intervention. Moreover, it may reside within the compartment for a substantial period of time, if desired (e.g., during a patient's stay in a hospital). Furthermore, removal is easy by withdrawing the contained fluid in the balloon end, and upon its collapse just pulling the catheter system. The puncture wound would be covered with just a small bandage and left to heal spontaneously.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

1. A compartment pressure monitoring device, comprising:
a tube connected at a first end to a balloon and at a second end to a pressure gauge or transducer; and
a trocar sleeve connected to the tube at or near its first end, wherein the pressure gauge or transducer includes an alarm device configured to sound or display an alarm, or both, when a predetermined level of pressure exerted on the balloon has been met or exceeded, or both.

2. The compartment pressure monitoring device of claim 1 in combination with a trocar.

3. The compartment pressure monitoring device of claim 2, wherein the trocar sleeve is configured to mechanically interact with the trocar.

4. The compartment pressure monitoring device of claim 1, wherein the predetermined level of pressure is about 40 mmHg.

5. The compartment pressure monitoring device of claim 1, wherein the predetermined level of pressure is about 30 mmHg.

6. The compartment pressure monitoring device of claim 1, further comprising a rigid support member connected to the tube at a point at or near the first end of the tube.

7. The compartment pressure monitoring device of claim 1, further comprising a fitting connected by an access tube to the tube at or near the second end of the tube, to provide fluid communication between the fitting and the tube.

8. The compartment pressure monitoring device of claim 7, wherein the fitting is a Luer lock type fitting.

9. The compartment pressure monitoring device of claim 7, in combination with a syringe that is configured to mechanically interact with the fitting to provide fluid communication between the syringe and the access tube.

10. The compartment pressure monitoring device of claim 1, wherein the tube is at least partially filled with a fluid.

11. A method of continuously monitoring pressure in a compartment, comprising:
inserting a portion of a compartment pressure monitoring device into a compartment of a patient, the compartment pressure monitoring device comprising
a tube connected at a first end to a balloon and at a second end to a pressure gauge or transducer, and
a trocar sleeve connected to the tube at or near its first end,
wherein the pressure gauge or transducer includes an alarm device configured to sound or display an alarm, or both, when a predetermined level of pressure exerted on the balloon has been met or exceeded, or both; and
using the compartment pressure monitoring device to continuously monitor the pressure within the compartment,
wherein the portion of the compartment pressure monitoring device that is inserted into the patient includes the first end of the tube, the balloon and the trocar sleeve.

12. The method of claim 11, wherein the compartment is an abdominal or pericardial compartment.

13. The method of claim 11, wherein the compartment is selected from the group consisting of a leg compartment, a forearm compartment, an upper arm compartment, a hand compartment, and a thigh compartment.

14. The method of claim 11, further comprising:
inserting the compartment pressure monitoring device into the compartment with a trocar,
wherein the trocar sleeve is configured to mechanically interact with the trocar.

15. The method of claim 14, further comprising removing the trocar from the compartment after inserting the portion of the compartment pressure monitoring device into the compartment.

16. The method of claim 11, wherein the predetermined level of pressure is about 40 mmHg.

17. The method of claim 11, wherein the predetermined level of pressure is about 30 mmHg.

18. The method of claim 11, wherein the compartment pressure monitoring device further comprises a rigid support member connected to the tube at a point at or near the first end of the tube.

19. The method of claim 11, wherein the compartment pressure monitoring device further comprises a fitting connected by an access tube to the tube at or near the second end of the tube, to provide fluid communication between the fitting and the tube.

20. The method of claim 19, wherein the fitting is a Luer lock fitting.

21. The method of claim 19, wherein the tube is at least partially filled with fluid.

22. The method of claim 21, further comprising adjusting the volume of the fluid in the tube by connecting a syringe to the fitting and mechanically operating the syringe.
